(19) United States
(12) Patent Application Publication
(10) Pub. No.: US 2007/0292894 A1
(43) Pub. Date: Dec. 20, 2007

(54) ASSAY METHOD

(76) Inventors: Miriam Victorine Dwek, London (GB); Anthony John Cullen Leathem, Oxfordshire (GB)

Correspondence Address: GREENLEE WINNER AND SULLIVAN P C, 4875 PEARL EAST CIRCLE, SUITE 200, BOULDER, CO, 80301, US

(21) Appl. No.: 11/779,120

(22) Filed: Jul. 17, 2007

Related U.S. Application Data
(63) Continuation of application No. 10/268,061, filed on Oct. 8, 2002, now abandoned.

(51) Int. Cl. G01N 33/573

(52) U.S. Cl. 435/7.4

(57) ABSTRACT

Detectable levels of fucosylation change in Prostate Specific Antigen when the prostate is cancerous, thereby allowing the reliable early detection of cancer of the prostate.

### FIELD OF THE INVENTION

The present invention relates to methods for the detection of a cancerous condition in the prostate, comprising assaying proteinaceous material associated therewith.

### BACKGROUND OF THE INVENTION

Various techniques exist for the detection of prostate cancer, but none of them, individually, is able to definitively diagnose the condition.

Cancer of the prostate is the second most common cause of cancer-related mortality among men [Hahnfeld L E, Moon T D (1999) Medical Clinical North America, 83(5), 1231-45] Because advanced disease is incurable, efforts have focused on identifying prostate cancer at an early stage, when it is confined to the prostate and therefore more amenable to cure. Unfortunately, prostate cancer can remain asymptomatic until tumor metastasis affects other organs or structures.

Symptoms associated with bladder outlet obstruction are commonly present in men over the age of 50 and are often ascribable to benign prostatic hyperplasia (BPH) and/or prostatitis. Neither of these conditions is immediately threatening. What is important is to accurately diagnose prostate cancer as early as possible, preferably avoiding the necessity for invasive testing.

Digital rectal examination (DRE) is a simple, inexpensive and direct method of assessing the prostate and has traditionally been considered the most accurate test for the detection of prostate cancer. However, DRE suffers from a lack of sensitivity, thereby yielding false-negative results and poor prediction rates. DRE is also known to have a relatively low specificity (69.7%) attributable to the test's inability to distinguish between benign and cancerous prostate conditions. Cancer detection rate increases when DRE is combined other methods of detection, such as transrectal ultrasound (TRUS) examination and/or prostate-specific antigen (PSA) analysis.

TRUS involves sonographic imagining of the groin, and has been useful in giving physicians the ability to “see” the prostate. However, its use in diagnosing prostate cancer is somewhat limited, owing to the fact that early prostate cancer is not visible by such techniques. The main use of TRUS is to provide an accurate guide for biopsy of the prostate.

After synthesis in the ductal epithelium and acini of the prostate, prostate specific antigen (PSA) is secreted into the lumina of the prostatic ducts to become a component of the seminal plasma [Wang et al. (1981) Prostate, 2, 89-96; Oesterling (1991) J. Urol., 145, 907-923]. PSA is a serine protease that exhibits proteolytic activity similar to chymotrypsin; it functions to liquefy the seminal coagulum that forms at ejaculation, thereby releasing the spermatozoa.

Studies have now clearly established that levels of PSA increase substantially in patients with advanced cancer of the prostate. Thus, a simple assay for PSA is useful as an indication as to whether a patient has prostate cancer.

Serum PSA levels have been shown to correlate generally with the volume, clinical state, and pathological stage of prostate cancer, although there is a wide range of PSA values associated with any given volume or stage. As noted above, PSA is secreted into the prostatic ducts, under normal circumstances but, when there is an obstruction, then PSA diffuses away from the site of secretion and can be detected at increased levels in the blood.

Unfortunately, the diagnostic value of PSA for prostate cancer is limited, due to its lack of specificity between benign and cancerous conditions [Egawa et al. (1999) Int. J. Urology, 6, 493-501]. As a result, benign conditions such as benign prostatic hyperplasia (BPH), prostatitis and infarction, as well as prostatic intraepithelial neoplasia, can be associated with elevated serum levels of PSA. In act, approximately two thirds of all elevated PSA levels (>4 ng/ml) in men over the age of 50 are due to BPH or prostatitis [Stenman et al. (1999) Cancer Biology, 9, 83-93]. Thus, merely establishing that a patient has elevated levels of PSA is not diagnostic of cancer, and further tests are necessary.

False-negative results are also common, since 20-25% of patients with early prostate cancer will have a normal PSA level (<4 ng/ml) and so cannot be accurately diagnosed using current technology [Mettlin et al. (1994) Cancer, 74, 1615-20]. Only one in four men who show a level of PSA in the so-called ‘grey-zone’ (a PSA level of 4 to 10 ng/ml) and, therefore, who are potentially curable, will actually have prostate cancer proven on biopsy.

Accordingly, assaying for elevated PSA levels has demonstrated itself to be an invaluable and relatively inexpensive means to provide an indication of early prostate cancer, but the unacceptable incidence of both false positive and false negative results has meant that a general screening program for potential prostate cancer sufferers cannot realistically be implemented.

Attempts to improve the accuracy of PSA testing have largely failed. Innovative prospects for PSA measurement include measuring PSA velocity [the rate of change of PSA level over time; Carter et al. (1992) Cancer Research, 52, 3323-28], PSA density [measuring the prostate volume via TRUS and dividing the volume by the serum PSA level; Benson et al. (1992) J. Urology, 147, 815-16; Zlotta et al. (1997) J. Urology, 157, 1315-21], age-adjusted PSA [the level of PSA increases with age and level of BPH; Partin et al. (1996) J. Urology, 155, 1336], and percent free PSA [Wang et al. (1996) Prostate, 28, 10-16]. This last approach exploits the observation that PSA exists in the blood in a free and bound form [Chu et al. (1999) J. Urology, 161, 2009-12]. Studies have shown that the proportion of free (unbound) PSA is decreased in some cases of prostate cancer when compared to cases of benign disease [Filella et al. (1997) Tumor Biology, 18, 332-40] None of these measurements, apart from age-specific PSA ranges, have been advocated for routine use. Percent free PSA may prove useful for staging prostate cancer, but further clinical trials are needed to ascertain the clinical usefulness [Polascik et al. (1999) J. Urology, 162, 293-306].

PSA is a single-chain glycoprotein consisting of 237 amino acids and is recognized as a member of the human kallikrein family. It has a molecular weight of about 28 kDa, including an N-linked biantennary oligosaccharide of approximately 2 kDa attached to Asp-45. There is evidence of an O-linked glycosylation site, although it is not clear whether this is occupied.

There have been a number of investigations into the oligosaccharide component of PSA. For instance, Glycobiology (2000), vol. 10 no. 2, pp. 173-176, teaches that PSA from normal prostate cells displays a biantennary oligosaccharide. This biantennary oligosaccharide from normal prostate cells is also fucosylated in around 70% of cases. Furthermore, it goes on to teach that cells from metastatic prostate carcinomas have bi-, tri- and possibly tetra-antennary oligosaccharides. Fucosylation is not discussed in the context of normal or cancerous condition of the cells.

The presence of fucose in glycoproteins from certain cancerous cells is known. EP-A-111,005 teaches that an antibody raised against a fucose-containing oligosaccharide [α-Fuc-(1-3, 1-4 or 1-6)-Gal] binds to mouse teratocarcinoma and human colon carcinoma cells. Cancer Research 55(1995), pp. 3654-3658, teaches that due to increased fucosyltransferase (FTase) activity, an increase in the levels of fucose was found in the tissues of endometrial carcinoma patients. However, it goes on to teach that FTase activity and, presumably, levels of fucosylation, is tissue dependent, as FTase activity was comparable in both normal and papillary serous carcinoma samples, so that the presence of fucose residues was not diagnostic.

Fucose-binding lectins have been used to show that entire tissue sections, from carcinoma prostate glands, featured increased levels of fucose [Glycoconj. J. (1999): Vol 16 (7), pp. 375-382]. The tissue sections were taken using techniques such as transurethral resections and prostatectomies. However, if possible, it is desired to avoid such invasive techniques.

Thus, there remains a need for a single, reliable assay for prostate cancer that does not rely on the use of invasive, surgical techniques.

Surprisingly, it has now been found that the PSA expressed by cancerous prostate glands has a higher, detectable level of fucosylation than that of PSA expressed by non-cancerous prostate tissue.

### SUMMARY OF THE INVENTION

Thus, in a first aspect, the present invention provides a method for the detection of a cancerous condition in the prostate gland of a male human subject, said method comprising:

obtaining a sample from the subject, the sample being of bodily tissue or fluid;

substantially removing cells and cell debris from the sample,

assaying for the presence of a glycosylated protein uniquely associated with the prostate in the sample;

and, where such protein is present in the sample, comparing fucosylation thereof with a standard control value indicative of a male human subject having a normal, non-cancerous prostate gland;

a level of fucosylation in the sample which is statistically significantly greater than that of the control being taken as indicative of the cancerous condition.

In a preferred embodiment, the present invention provides a method for the detection of a cancerous condition in the prostate gland of a male human subject, said method comprising:

obtaining a sample from the subject, the sample being of bodily tissue or fluid;

substantially removing cells and cell debris from the sample,

assaying for the presence of Prostate Specific Antigen (PSA) in the sample;

and, where PSA is present in the sample, comparing fucosylation thereof with a standard control value indicative of PSA from a male human subject having a normal, non-cancerous prostate gland;

a level of fucosylation of PSA in the sample which is statistically significantly greater than that of the control being taken as indicative of the cancerous condition.

### BRIEF DESCRIPTION OF THE DRAWINGS

FIG. 1a shows Western Blots of PSA stained with anti-PSA antibody and Ulex europaeus lectin from both Benign Prostatic Hyperplasia (BPH) and cancerous prostatic tissues. FIG. 1b compares the relative density of two proteins between the cancerous and the hyperplastic tissues.

FIG. 2 shows a biopsy sample containing both cancerous and normal tissue, stained with lectin, ‘Ca’ indicates invasive cancer cells whilst ‘N’ indicates normal prostate epithelial cells.

FIG. 3 shows a densitometry profile of Ulex staining, taken from the marked rectangular box in FIG. 2. ‘Ca’ indicates cancer cells, ‘N’ indicates normal epithelial cells and ‘B’ indicates the background level.

FIG. 4 shows the result of staining samples of semen that have been diluted 1 in 200. FIG. 4a shows the result of silver staining whilst FIG. 4b shows the result of anti-PSA staining. Lanes 1 and 12 contain a molecular weight marker, Lane 2 contains semen as applied to the column; Lane 3 contains semen as passed through the column; Lanes 4 to 11 contain specifically eluted PSA fractions (1 to 8).

FIG. 5 shows the results of staining semen diluted 1 in 100,000. FIG. 5a shows the results of staining with silver stain, whilst FIG. 5b shows the results of staining with anti-PSA stain. Lanes 1 and 12 contain molecular weight markers; Lanes 2-11 contain the eluted PSA fractions 1 to 10.

FIG. 6 shows the results of staining serum diluted 1 in 5. FIG. 6a shows the results of staining with silver stain; Lane 1 contains molecular weight markers and Lanes 2-6 contain eluted PSA fractions 1 to 5. FIG. 6b shows the results of staining with anti-PSA stain. Lane 9 contains molecular weight markers and Lanes 1-8 contain the eluted PSA fractions 1 to 10.

FIG. 7 shows the results of staining PSA isolated from serum of a patient with cancer. FIG. 7a shows the results of staining with lectin stain, whilst FIG. 7b shows the results of staining with anti-PSA stain. In both figures, Lane 1 contains molecular weight markers and Lane 2 contains PSA isolated from a patient with prostate cancer.

FIG. 8 shows the results of staining PSA isolated from serum of patient with BPH. FIG. 8a shows the results of staining with lectin stain, whilst FIG. 7b shows the results of staining with anti-PSA stain. In both figures, Lane 1 contains molecular weight markers and Lane 2 contains PSA isolated from a patient with prostate cancer.

FIG. 9 shows the mean relative intensity of UEA/PSA for the prostate cancer group, BPH patients, and those who have received treatment for their prostate cancer.

### DETAILED DESCRIPTION OF THE INVENTION

Whilst it has been found that fucosylation of PSA apparently increases with cancerous conditions, this also appears to be the case with other prostatic glycoproteins, and is exceptionally useful in assuring early detection of cancer in those sections of the population deemed susceptible.

It will be appreciated that “a glycosylated protein uniquely associated with the prostate” is one which is associated with the prostate, and only the prostate, thereby ensuring that any abnormality can be attributed directly to the prostate, rather than having to conduct any further tests.

The protein may be assayed as is, or fragments of the protein may be assayed. The protein may degrade in vivo, so that the assay of the invention necessarily involves detecting and assaying fucosylation levels of any relevant fragments, or the sample may be so treated as to disrupt the protein for easier assay, for example. Thus, it will be understood that reference to PSA or a protein uniquely associated with the prostate includes reference to any fragments thereof, especially where such fragments include fucosylation sites.

The method of the present invention may be performed on any sample containing, or likely to contain, the glycosylated protein, as well as samples unlikely to contain the protein, as negative controls, if desired. It will be readily apparent to those skilled in the art how such samples may be obtained. In general, blood is the easiest to sample and assay, but seminal fluid or urine may also be used. The latter two may give rise to misleading results in the case of PSA, however. Other samples, such as serum or prostate biopsy sample, may also be used, as well as any tissue or bodily fluid likely to contain indicative amounts of the antigen. As used herein, the terms “glycosylated protein” and “PSA” are interchangeable, except where otherwise apparent.

Levels of fucosylation greater than those associated with a normal healthy prostate can be taken as indicative of or, at higher levels, diagnostic of, a cancerous condition in the prostate of the subject. The control for the method, or assay, of the invention is generally one which can be taken as indicative of a healthy prostate. This may vary within certain tolerances, but the levels of fucosylation associated with cancer are generally substantially in excess of any reasonable tolerance in healthy levels of fucosylation, as illustrated in the accompanying Figures and Examples.

Although it is not essential to the present invention, it is preferred that the method comprises an assay to determine the level of PSA in the sample. This may be done contemporaneously, before or after assaying fucosylation. However, it is preferred to assay PSA levels prior to assaying fucosylation.

It will be appreciated that assaying levels of PSA in the sample can be used, as known in the art, to provide some indication of the possibility of cancer in the prostate. Assaying levels of PSA also serves to calibrate the results of the fucosylation assay.

Although it is not essential to the present invention, it may be advantageous to at least partially purify the sample. This becomes necessary where there is any likelihood of cellular contamination, especially where lectins are used to determine the presence of the protein, or PSA, in which case a purification step, such as filtration or centrifugation is desirable. Especially in the case of biopsy, the sample may be suspended in buffer and, if necessary, homogenized, for example. The buffered sample may then be centrifuged to remove any detritus, and the supernatant may then be used, as is, for assaying.

However, fucosylation is quite common in various biological samples, so that it may well be appropriate to run the purified sample on a gel, before assay. Should it be desired, the sample may further be concentrated by any suitable method, such as column chromatography or SDS-PAGE electrophoresis. Too much purification of the sample adds expense to the procedure and may make the method of the invention impractical for use in mass screening of the susceptible portion of the population.

Antibodies, especially monoclonal antibodies, specific for PSA, but insensitive to fucosylation levels, are well known, and are readily commercially available. PSA-specific antibodies may be bound to a support, in order to fix the PSA in a sample, or may be suitably labeled for use in an appropriate blotting technique. The label may be any that is appropriate, including direct and indirect labeling. An example of direct labeling is isotopic labeling, so that any antibody present in the sample can be detected by autoradiography or scintigraphy, after removal of unbound antibody. An example of indirect labeling is enzyme labeling, such that, after removal of unbound antibody, bound antibody can be detected by reaction with a suitable substrate. It is preferred that the reaction with the substrate is readily detectable, such as a chromogenic reaction.

A preferred detection method is to use PSA-specific antibody bound to a plate such as a microarray, for example. A blood sample can then be exposed to the plate for a suitable period and then washed off. PSA will be left, bound to the antibody, and levels of fucosylation can then be detected and compared with a PSA standard known to be sampled from a non-cancerous, normal, healthy prostate. Alternatively PSA bound in such a way can be washed off, collected and run on a gel, and fucosylation assayed by a suitable blot.

In general, if the amount of PSA in the sample is known, then it may be sufficient merely to assay the level of fucosylation in order to provide an indication of the presence or absence of cancerous tissue. However, for greater accuracy, it is generally preferred to partially purify the sample, as described above, and stain with a fucose-specific binder.

In particular, we have established that fucosylation of PSA from benign tissue shows up as discrete bands, for instance in a suitable blot, whereas PSA from cancerous tissue shows clusters of bands bound by the fucose-specific binder. It is these clusters of bands which show up as higher levels of fucosylation and which are diagnostic of prostate cancer.

Any suitable substance may be used as the fucose-specific binder, but we have found that certain lectins are preferable. For example, a lectin such as Ulex europaeus I (UEA-1), isolated from gorse, or Lotus tetragonolobus lectin, isolated from winged peas, may be used to identify fucose moieties within the oligosaccharide.

Ulex europaeus agglutinin 1 is a glycoprotein with a molecular weight of 63,000, although multimeric aggregates have been reported. UEA-1 has two subunits, one of about 31,000 Daltons and another of 32,000 Daltons, and binds to many glycoproteins and glycolipids containing α-linked fucose residues, such as ABO blood group glycoconjugates. This lectin preferentially binds blood group O cells and has been used to determine secretor status. It has been established as an excellent marker for human endothelial cells. For these reasons, it is important that cells and cell debris be removed from any sample before assaying with UEA-1.

Lotus tetragonolobus lectin is a family of closely related glycoproteins having 2 or 4 subunits of about 28,000 Daltons. These isolectins have similar specificities toward α-linked L-fucose containing oligosaccharides. Although many of the binding properties of Lotus lectin are similar to those of UEA-1, the binding affinities and some specificities for oligosaccharides are markedly different.

Any suitable form of labeling may be employed for the lectins, or the lectins may be detected by an antibody specific for the lectin. If the lectin is labeled, then it is preferred that the labeling should produce a color or light, or be isotopic in nature.

In 1989 a study was carried out by Abel et al. investigating the lectin Ulex europaeus (UEA) binding to human prostatic epithelium. Immunoperoxidase techniques were used to stain formalin-fixed, paraffin-bedded sections of prostate tissue from men with benign prostatic hyperplasia and with carcinoma of the prostate. The study found that in benign epithelium less than 10% of cells stained with UEA1, however in malignant epithelium more than 90% of cells stained with UEA1 (Abel, P. D., et al., British Journal of Urology, 1989; 63: 183-185). These findings suggested that the glycosylation of glycoproteins expressed by the prostatic epithelium were altered during malignant transformation of the prostate.

It will be readily apparent that the nature of PSA from cancerous tissue is different from that of PSA from benign tissue. In particular, we have established that more fucose residues are detectable. However, the altered PSA is still detectable by standard PSA detection techniques, and the actual nature of the PSA is unimportant to the present invention. All that is necessary is to assay fucosylation of a sample known to contain PSA.

Experiments have shown that there is an abnormality in the oligosaccharide linked to PSA. Detectable levels of fucosylation of PSA are apparent, but may be attributable to increased fucosylation, or increased accessibility of fucose residues, or both.

Although the oligosaccharide component of cancerous PSA has yet to be fully sequenced, and without being bound by theory, it appears that the abnormal glycan displayed on cancerous PSA is a truncated version of the bi-antennary fucosylated glycan on normal PSA. The truncation appears to remove the terminal NeuNAc residue, thus exposing a fucose on one arm of the antennae, to which the Ulex lectin may bind. Alternatively the Ulex lectin may bind to a Fucose on the first GlcNAc residue. In either case, the Ulex lectin, in addition to binding to Fucose nay also bind to a GlcNAc residue. It will be apparent that reference to Ulex lectin is equally applicable to any substance useful in the context of the present invention to bind the fucose residues of cancerous PSA, and any other reference herein to Ulex lectin should also be so understood.

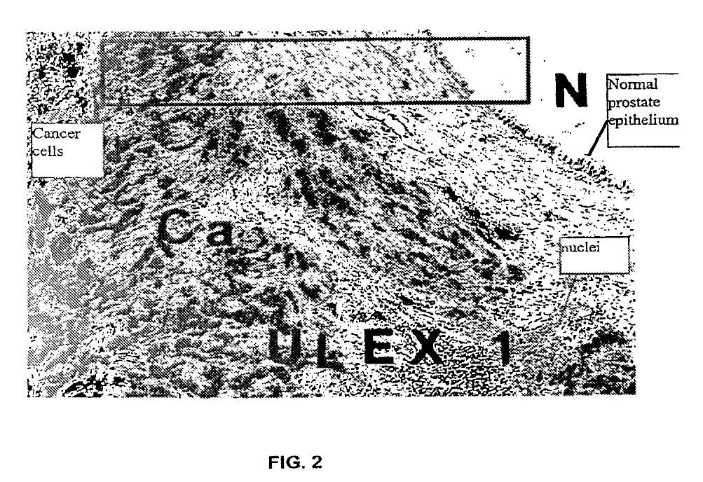

It is also possible that the Ulex lectin, due to steric changes in the oligopeptide resulting from truncation, may be able to both bind fucose and interact hydrophobically with a number of adjacent amino acids, as shown in FIG. 2 above.

Alternatively, entirely new glycosylation sites may be involved in binding the Ulex lectin, including fucosylated O-linked glycans.

In another embodiment, the assay method includes isolating PSA having an abnormal fucose content, contacting said isolated PSA with a binding molecule and detecting binding of the binding molecule. Preferably the binding molecule is an antibody. Alternatively, the binding molecule may be a lectin. In yet another embodiment, PSA having an abnormal fucose content may be isolated by an antibody before being contacted with a binding molecule, such as a second antibody or a lectin, and detecting binding of the binding molecule. While lectins are known to show a binding affinity for fucose, it will be appreciated that other chemical entities which show a suitable binding affinity may be used.

Before, or after, the bound PSA is contacted with the labeled binding molecule, it may be necessary to separate the free binding molecule from the bound binding molecule. The presence of PSA having an abnormal fucose content may then be determined by means known to those skilled in the art and depending on the label or marker employed. In this respect, it is preferred if the marker is by means of the appearance of a color or a real color change to enable quick and accurate interpretation of the assay.

Other assay systems may include semi-automated or fully automated detectors, including biosensors.

In yet another aspect, the present invention provides a kit for testing a sample derived from a mammal comprising means for detecting PSA having an abnormal fucose content.

Preferably, the kit comprises a substance which binds to the PSA and a means of detecting the bound substance.

To maximize specificity of the test it is advantageous that the substance is tailored to detect an abnormal fucose content in the oligosaccharide linked to the PSA.

Conveniently, the means of detecting bound substance may be by way of a color change which may be brought about, for example, by an enzyme reaction activated by binding of the substance to the PSA. The color change is preferably evaluated by the eye, but may be measured by a photometer, fluorescence detector, refractive index detector, or any chemico-physico detector including a radioactive isotope detector.

In another embodiment, the kit may comprise a substance which isolates PSA, another substance which recognizes an abnormal fucose content associated with the PSA, and means for detecting binding of either or both substances.

In a further embodiment, the kit may comprise a first substance which binds to PSA, a second substance which recognizes and binds to an abnormal fucose content associated with the oligosaccharide linked to the PSA, and means of detecting binding of the second substance. In this way, PSA is isolated from the sample before the abnormal fucose content is identified in the PSA.

It will be appreciated that PSA may be isolated by numerous methods which are well known to those skilled in the art, such as affinity chromatography and/or HPLC and/or gel filtration or SDS PAGE. Accordingly, anyone of these methods may also be deployed in the present invention.

Alternatively, PSA may be isolated through the use of a suitable chemical agent which binds to a part of PSA or the linked oligosaccharide having an abnormal fucose content.

The present invention also provides an antibody which binds to prostate specific antigen (PSA) having an abnormal fucose content. The antibody may be polyclonal or monoclonal, but preferably is monoclonal.

Studies have suggested that PSA may be involved in prostate cancer progression, by modulating cell growth and mediating invasion of prostate cancer (Stenman, U., et al., Seminars in Cancer Biology, 1999; 9: 83-93). Thus, over-fucosylated PSA identified in accordance with the present invention may serve as a suitable antigen. Monoclonal antibodies specific for over-fucosylated PSA and which do not recognize naturally occurring PSA form a further aspect of the present invention, and are suitable for the treatment of prostate cancer. These are useful in the treatment of prostate cancer and in the slowing or stopping of cancer invasion.

As shown below, lectins binding fucose residues bind particularly strongly to cancerous prostate tissue, but do not bind at all well to benign tissue from the same sample. Accordingly, there is further provided an assay method comprising detecting abnormal fucosylation in a biopsy sample. Such an assay may suitably employ any of the techniques described herein in relation to other forms of assay.

Given that the sequence of normal PSA is known, having elucidated the sequence of the cancerous PSA oligosaccharide, normal PSA can be readily treated to resemble cancerous PSA. For instance, normal PSA may be enzymatically treated with suitable enzymes such as glycanases or transferases, so that the oligosaccharide component is altered to have the same sequence and structure as that of cancerous PSA.

Normal PSA is readily available, for instance from semen, and can be easily isolated, for instance by affinity chromatography using an immobilized anti-normal PSA antibody. Since there is a ready supply of normal PSA, it follows that, by using of enzymic alteration, for example, a large amount of cancerous-type PSA can be quickly and easily obtained. Alternatively, as the peptide and the oligosaccharide components of the oligopeptide can be sequenced, the oligopeptide can be synthesized or produced by other biotechnological methods known in the art, such as expression in bacterial systems. In addition, cancerous-type PSA may be obtained directly from patients.

Once a suitable quantity of cancerous-type PSA had been obtained, it is readily possible to raise antibodies thereagainst, using a suitable mammal, such as mouse or rabbit. Antibodies specific for the cancerous form of PSA may be obtained by eliminating clones recognizing normal PSA. Such antibodies, preferably humanized by methods well known in the art, may be used in therapy. It will be appreciated that humanization is unnecessary if the antibodies are for use simply in the diagnosis of prostate cancer. Such antibodies may also be used as an immunogen for use in immunotherapy, their anti-idiotypes generating suitable anti-ant-idiotypes reactive against and/or specific for cancerous PSA.

Cancerous PSA may also be treated with proteases to produce cancerous PSA peptide fragments. Those fragments having fucosylation characteristic of a cancerous condition are considered as cancerous PSA glycopeptides. Antibodies may preferably be raised against any of such PSA glycopeptides. Such anti-cancerous PSA glycopeptide antibodies are generally more suitable than Ulex lectin in diagnosis, as not only may they be produced relatively cheaply and in greater quantity than Ulex lectin, but they are generally specific for cancerous PSA, and nothing else.

In a further embodiment of the present invention, anti cancerous PSA glycopeptide antibodies are further used to develop a protein mimic or anti-idiotype of cancerous PSA. Such mimics or anti-idiotypes may then be used in immunotherapy. By raising flier antibodies to an anti-cancerous PSA glycopeptide antibody and screening which of these anti-anti-cancerous PSA glycopeptide antibodies bind to the binding site of the anti-cancerous PSA glycopeptide antibody, an anti-idiotype of the cancerous PSA glycopeptide may be identified. The screening process may suitably involve screening which of the anti-anti-cancerous PSA glycopeptide antibodies prevent anti-cancerous PSA glycopeptide antibody from binding cancerous PSA glycopeptide, thereby indicating that the anti-anti-cancerous PSA glycopeptide antibody is competing with the cancerous PSA glycopeptide by binding at, or near to the anti-cancerous PSA glycopeptide antibody binding site for the cancerous PSA glycopeptide.

The antibodies described above may be monoclonal or polyclonal, but are preferably monoclonal.

Once isolated, the amino acid and, therefore, the DNA sequence of the anti-idiotype mimic is readily determined. Where the antibody is for administration to a human subject, then it is preferable that this information be used in a well known humanization technique. This reduces or eliminates any likelihood of an immune reaction against the foreign antibody, other than an anti-idiotype response. It will be appreciated that, due to the degeneracy of the genetic code, it is preferred that the DNA sequence that codes for the anti-idiotype mimic is elucidated from the host organism in which it was raised. The techniques for identifying and elucidating the sequence of a protein of interest are well known in the art.

The DNA corresponding to the anti-idiotype mimic can then be used for immunotherapy. It may be used as an immunogen to develop further antibodies for diagnosis, or it may expressed in an individual in order to stimulate an immune response in that individual to the anti-idiotype mimic and, therefore, the cancerous PSA glycopeptide itself.

The DNA encoding the anti-idiotype mimic peptide can be expressed in an individual, for instance, a human, using gene transfer techniques that are well known in the art. For instance, a plasmid or vector containing the DNA can be introduced by transfection techniques such as electroporation, microinjection, use of a microprojectile accelerator or particle gun, lipofection, co-precipitation or the use of polycations such as DEAE-dextran. Preferably, however, a viral vector, for instance a retroviral vector or an adenoviral vector, can be used. For example, a vector comprising adeno-associated virus (AAV) can be used

The cells expressing the protein mimicking a PSA peptide, preferably the cancerous PSA glycopeptide may be in an in vitro cell culture which may be subsequently introduced into an individual. Alternatively these cells may already be present in an individual, for example, a human.

Thus, it will be appreciated that the DNA of the anti-idiotype mimic may be used to develop an anti-cancer treatment, for example a vaccine. For instance, a vaccine could be produced using gene transfer techniques, so that a protein mimicking a PSA peptide, preferably the cancerous PSA glycopeptide, is introduced into any number of cells in vivo. The anti-idiotype mimic produced by these cells would trigger the individual's immune system to mount an anti-cancer immune response.

Therefore, in a further aspect of the present invention, there is provided a system comprising a vaccine, that produces a peptide which mimics a cancerous PSA peptide, preferably the cancerous PSA glycopeptide displaying the cancer-associated oligosaccharide. Preferably, this system will elicit an anti-cancer immune response in the individual. Preferably, the antigenic site will be suitably immunogenically presented, such as by selecting immunogenic flanking regions.

The invention will now be illustrated by way of the following example which is not intended to limit the scope of the invention in any way.

### EXAMPLE 1

Prostate chipping samples were taken from patients having histologically confirmed prostate cancer or benign prostatic disease, and stored at −80° C. until required. Before use, the samples were thoroughly defrosted before being homogenized in a solution of de-ionized water and centrifuged. The resulting supernatant was subjected to SDS-PAGE followed by ECL™ Western Blotting (Amersham Life Science). Immunostaining of the blots using α-PSA polyclonal antibody (The Binding Site Ltd., UK) was carried out to identify and estimate any changes in the molecular mass of PSA. Alternatively, gels were stained with the lectin UEA-1 (Sigma, UK) to ascertain any differences between the two sample groups due to alterations in fucosylation.

Analysis of the resulting autoradiography films revealed a protein corresponding to PSA having a molecular weight of about 30 kDa in all samples, thereby suggesting no difference in the molecular weight of PSA between the benign and carcinomic tissue. However, UEA-1 binding to benign tissue samples produced discrete bands whereas UEA-1 binding to carcinomic tissue samples produced clusters of bands, perhaps indicating altered fragmentation of PSA and its associated oligosaccharide. Since UEA-1 binds to fucose-containing oligosaccharides, these results suggest a cancer-associated alteration in fucosylation of the PSA glycoprotein.

### EXAMPLE 2

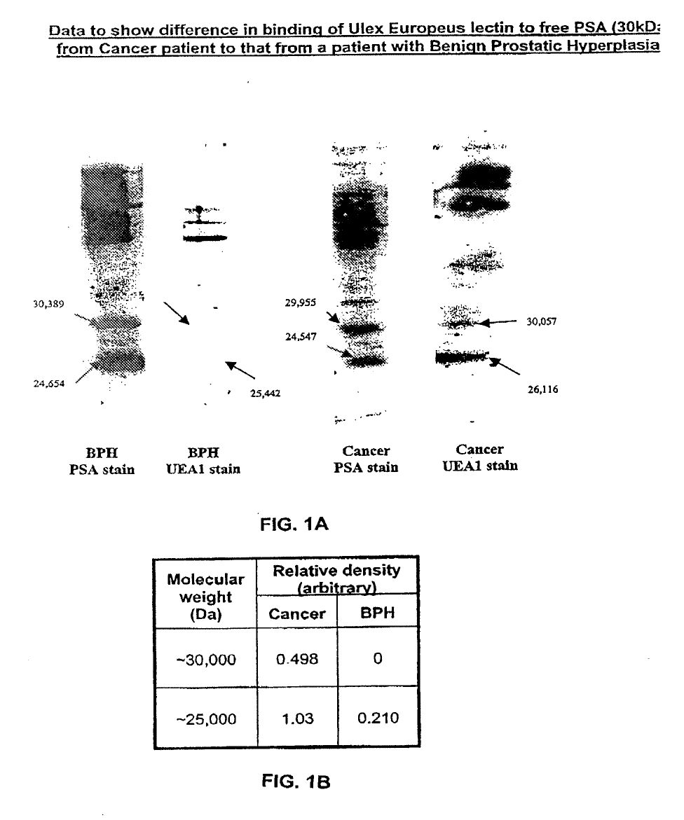

The results shown in FIG. 1a were taken from Western blots. PSA was isolated from serum of a patient with prostate cancer and a patient with benign prostatic hyperplasia. The PSA was then run on SDS-PAGE, Western blotted and then stained with anti-PSA antibody and Ulex europaeus lectin. Binding was detected by enhanced chemiluminescence (ECL), and the results are shown below. The figures were obtained by scanning the ECL results and using a program (Bandscan from Glyko), to measure the density of the different bands. The program measures density on the basis of “peak grey”. The densities of the bands were measured and divided by the corresponding anti-PSA stained band, in order to eliminate discrepancies caused by the different concentrations of PSA in the bands. This also enabled comparison between gels, FIG. 1b.

### EXAMPLE 3

A biopsy sample containing both cancerous and normal tissue was stained with lectin. The results are shown in FIG. 2.

Ulex lectin staining of normal prostate and of prostate cancer in the same tissue sample. Paraffin section, magnified circa ×250, showing strong binding of Ulex, as seen by large, diffuse, black/grey staining, to invasive cancer cells (Ca) and weak staining, slightly higher than background, to the normal prostate epithelial cells (N). To assist identification of cells, the nuclei of all the cells were lightly stained with haematoxylin, seen as discrete grey dots (wowed nuclei).

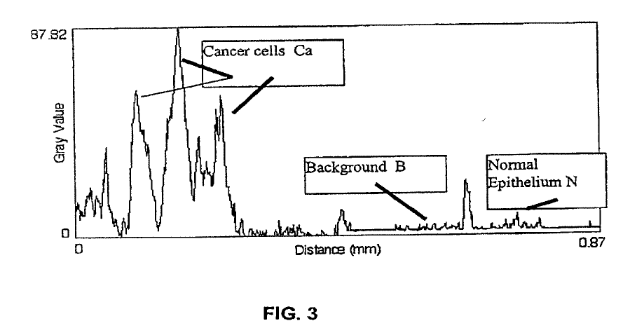

FIG. 3 shows a densitometry profile of Ulex staining, taken from the marked rectangular box in FIG. 2. The profile shows cancer cells binding Ulex many times higher (approx. 5-10×) on cancer cells (Ca) than on normal epithelium (N) or ‘background’ level (B).

Fucose-related structures are, thus, over-expressed in prostate cancer cells, probably on many glycoprotein products, including PSA, Prostatic Acid Phosphatase, and other serum markers.

### EXAMPLE 4

### Methods and Materials

Preparation of affinity columns for extraction of PSA: An anti-PSA antibody was conjugated to a Sepharose matrix, via a cyanogen-bromide linkage. The method for conjugating the antibody to the beads was adapted from a book from Pharmacia called Affinity Chromatography Principles and Methods, published in 1979. The required amount of cyanogen-bromide activated Sepharose 4B powder was measured to give a column volume of 0.5 ml. The cyanogen-bromide activated Sepharose 4B beads (Sigma-Aldrich, Poole, UK) were swollen for 15 minutes at 4° C. in 1 mM HCL, and washed on a sintered glass filter, approximately 200 ml of HCL per gram of beads was added in several aliquots, the supernatant being removed between successive additions. HCL is used to swell the beads as it preserves the activity of the reactive groups that hydrolyze at high pH. The beads were then washed with coupling buffer (0.25 M NaHCO₃, 0.5 M NaCl, pH 9.0), 5 ml buffer per gram of beads and transferred immediately to a solution containing a polyclonal rabbit anti-human PSA antibody (Dako, Cambridge, UK) diluted in coupling buffer, 2 mg antibody per ml of beads. A gel to buffer ratio of 1:2 was used for coupling and the solution was mixed for two hours at room temperature on an end-over-end mixer. After this period the coupling buffer was removed and any residual active groups on the beads were blocked with 0.2 M glycine pH 8.0 for two hours at room temperature. The beads were then washed alternately with coupling buffer followed by 0.1 M acetate buffer, pH 4.0 for 5 cycles on a sintered glass filter. A change in pH causes protein desorption, removing non-specifically bound antibody from the matrix. The conjugated beads were transferred into Polyprep columns (Biorad, Hertfordshire, UK) and equilibrated with phosphate buffered saline, pH 7.4 (PBS) and stored at 4° C. until used.

Extraction of PSA using affinity chromatography. The columns were optimized using semen; the concentration of PSA in the semen is approximately 2.5 mg/ml (between 0.5 and 5 mg/ml) (Noldus et al, 1997), therefore it was ideal for testing the sensitivity of the columns. Semen was collected and centrifuged for 30 minutes at 3000 rpm; the supernatant removed and diluted to test the sensitivity of the column. The semen samples were diluted with PBS to reflect amounts of PSA in the serum. Dilutions of 1 in 200 and 1 in 100,000 were prepared and passed through the affinity chromatography column. The best results were achieved by passing the semen through the affinity matrix three times with a flow rate of approximately 0.4 ml/min. The columns were capped between each run to allow time for the reaction between the antigen and the antibody on the column matrix. Time periods of 30, 45 and 60 minutes were investigated and a period of 45 minutes was found to be sufficient to allow successful binding and keep non-specific binding to a minimum. Unbound material was removed by washing the matrix with 20 column bed volumes of PBS. The PSA was eluted with 0.1 M Glycine—HCL pH 2.5, containing 0.5M NaCl, at a flow rate of approximately 0.4 ml/min. Fractions (0.5 ml) were collected and neutralized with 80 μl of 1 M Tris pH 8.0, to protect the protein against denaturation by the low pH of the glycine-HCL buffer.

Serum preparation Serum was available for use in this study having, been collected from patients with prostate cancer and patients with benign prostatic hyperplasia from Urology clinic at Middlesex Hospital, London. Normal serum was collected with artificially raised PSA levels. Raised PSA levels can be achieved in two ways: by prostatic massage or by taking the blood within 24 hours of ejaculation/sexual activity, of which the latter applied herein. The serum was stored at −80° C. until ready for use. Prior to running through the column the serum was centrifuged at 3000 rpm for 30 minutes, the supernatant then diluted with PBS (1 in 5) and filtered through a 1 μm membrane filter (Whatman, Kent, UK) to clarify it. A dilution of 1 in 5 was found to bc most effective in that it gave a small (5 ml) sample volume and non-specific binding was found to be minimal. The serum was passed over the affinity matrix in the same way as described with semen and the fractions were run on SDS-PAGE as described previously.

Quality control of affinity chromatography columns: The fractions collected from the affinity chromatography were subjected to sodium dodecyl sulfate polyacrylamide gel electrophoresis (SDS-PAGE) through 10% T 3% C separating gel under reducing conditions using an ATTO AE-6450 vertical electrophoresis system. Reducing conditions were achieved by mixing the fraction 1:1 with sample buffer (4% SDS, 20% glycerol, 10% 2-mercaptoethanol, 0.004% bromophenol blue, 0.125 M tris-HCL, pH6.8) and boiling for 3 minutes. Gels were electrophoresed at 165V for 1 hour 15 minutes, using tank buffer of 25 mM Tris, 192 mM Glycine and 0.1% SDS, pH 8.3. Two identical gels were electrophoresed in the same tank, one for Western blotting and the other for silver staining.

Western blotting: The proteins from the gels were transferred to pre-wetted nitrocellulose (Biorad, Hertfordshire, UK) by semidry-electrophoretic transfer using an SV20 semi-dry blotter (Sigma-Aldrich, Poole, UK) at 2.5 mA/cm² of gel for 30 minutes at room temperature. Conditions for blocking and immunodetection (buffers used, antibody concentration and incubation times) were established in previous experiments. Blots were blocked overnight with agitation at 4° C. in 2% w/v bovine serum albumin (BSA) in tris buffered saline, pH 7.6 containing 0.1% v/v Tween-20 (TBS-T). A polyclonal rabbit anti-human PSA antibody, 8 μg/ml (DAKO, Cambridge, UK) was used as the primary antibody for two hours to probe PSA, a biotinylated polyclonal swine anti-rabbit immunoglobulins antibody, 1.625 μg/ml (DAKO, Cambridge, UK) was used as a secondary antibody for two hours to amplify the detection of the PSA and Streptavidin conjugated to horse radish peroxidase, 1.8 μg/ml (DAKO, Cambridge, UK) was used for one hour to label the PSA. All incubations were carried out at room temperature with agitation, the blots were washed with TBS-T five times for five minutes between incubations, finally washed three times for five minutes with TBS-T and three times for five minutes with TBS prior to developing. The blots were developed using an Emission Chemiluminescence (ECL) kit according to the instructions of the manufacturer (Amersham Pharmacia Biotechnology, Buckinghamshire, UK). The PSA bands were visualized by exposing to ECL Hyperfilm (Amersham Pharmacia Biotechnology, Buckinghamshire, UK) for 30 seconds and 1 minute.

Silver staining: The other identical gel was stained for total protein using a silver stain kit (Insight Biotechnology, Middlesex, UK) according to the manufacturers instructions. The result of this stain should reveal the extent of non-specific binding to the affinity column.

Lectin Staining: The PSA isolated from each serum sample was run on SDS-PAGE in the same way as described and blotted to nitrocellulose, this time the PSA was run under non-reducing conditions. Two identical gels were electrophoresed in the same tank and the proteins transferred to nitrocellulose at the same time, one blot was probed for PSA using the immunostaining method already described and the other blot was probed with biotinylated Ulex europaeus (Sigma-Aldrich, Poole, UK). For this the blot was blocked overnight with agitation at 4° C. in 2% bovine serum albumin in TBS-T, incubated in the biotinylated UEA1 for two hours at a concentration of 5 ug/ml. The lectin was detected by incubation with horseradish peroxidase (HRP) conjugated Streptavidin 1.8 μg/ml for 1 hour, then the ECL reaction undertaken. All incubations were carried out at room temperature and with agitation Again the blots were washed for five times for five minutes with TBS-T between incubations and finally three times for five minutes with TBS-T followed by three times for five minutes with TBS. The X-ray film was developed manually by immersing in developer (Photosol, UK) for 30 seconds, dipped briefly in water and fixed in fixative (Photosol) for 90 seconds. The developed ECL X-ray films were scanned using a calibrated imaging densitometer, GS-710 (Biorad, Hertfordshire, UK), into the data analysis program Quantity One (Biorad, Hertfordshire, UK), which was used to measure the intensity of each of the stained bands. The program was used to calculate molecular weight values using the Rf values of proteins in a molecular weight marker (Sigma-Aldrich, Poole, UK) that was run along side the fractions. The intensity of the free PSA band (28-30 kDa) was measured from both the X-rays. The intensity of the UEA1 stained band was divided by the intensity of the PSA stained band, to eliminate discrepancies caused by the different concentrations of the PSA in the bands and to enable a comparison of UEA1 binding in different samples run on different gels.

Results:

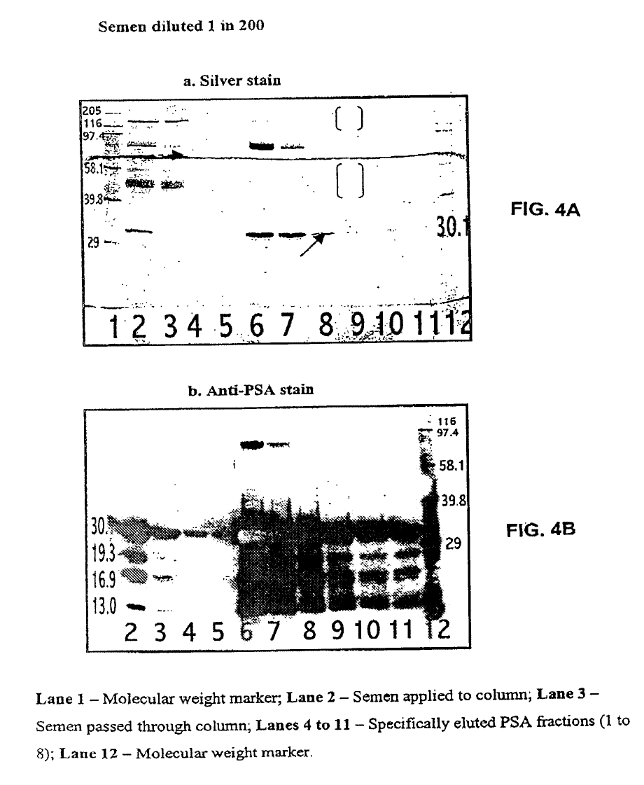

Affinity Chromatography for isolation of PSA: The results of isolating PSA, using the affinity columns prepared, from semen diluted 1 in 200, are shown in FIG. 4.

In FIG. 4, semen has been diluted 1 in 200. FIG. 4 a shows the result of silver staining whilst FIG. 4b shows the result of anti-PSA staining. Lanes 1 and 12 contain a molecular weight marker; Lane 2 contains semen as applied to the column; Lane 3 contains semen as passed through the column; Lanes 4 to 11 contain specifically eluted PSA fractions (1 to 8).

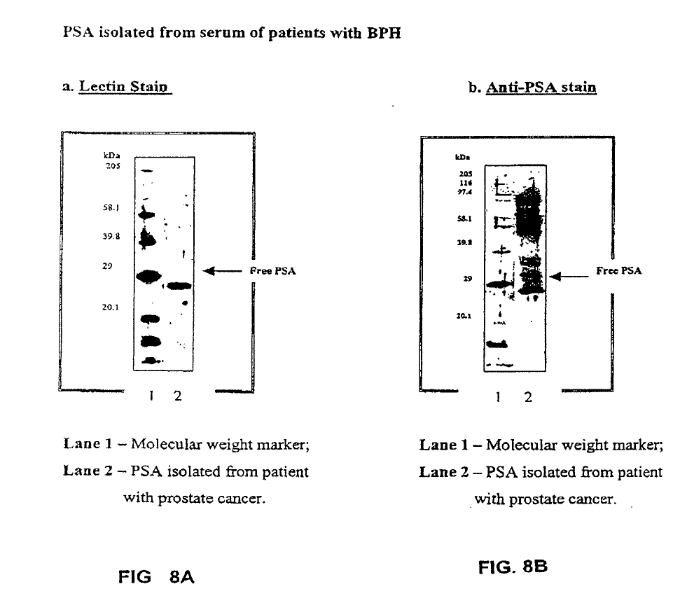

FIG. 4a shows the results of the silver stain, a clear band at 30 kDa was detected in the eluted fractions (see black arrow); immunostaining with an anti-PSA antibody, identified this band as PSA (see black arrow). This suggested that the columns had been successful at isolating PSA. Lane 6 of FIG. 4a shows that there were some non-specific proteins in some of the fractions, in that there were bands present in the silver stain, which were absent on the anti-PSA stain (see brackets). However the intensity of the non-specific bands was considerably lower than the bands identified as PSA. FIG. 4b shows the ECL result of the anti-PSA stain, the free PSA band at 30 kDa has stained white (see black arrow), this phenomenon is called “negative staining”, which is due to the amount of PSA in this band being too high for detection. Several bands with lower molecular weights than free PSA also stained intensely, according to their molecular weights these are most probably clipped forms of PSA. In semen 30% of PSA exists in the clipped form (Mikolajczyk et al, 1997). A higher molecular weight band at about 90 kDa was observed in FIGS. 8a and 8b, this may represent PSA bound to another seminal protein. Lane 1 shows the diluted semen that was applied to the column and lane two shows the semen that came through the column; the results show that there is a clear decrease in the band at 30 kDa. However the anti-PSA immunostain shows that there was still some PSA left, which did not bind to the affinity column, indicating that it was necessary to put the semen through the column more than once to isolate more PSA.

The band seen stretching across FIG. 4a (dashed arrow), was consistent with all silver stains and was assumed to be due to a reaction between buffers within the gel and the silver stain kit, and was therefore ignored.

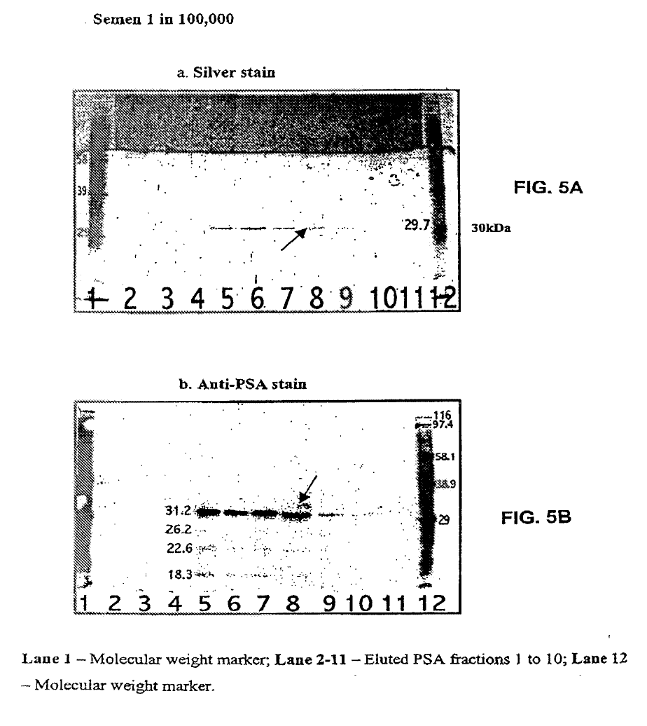

Assuming that the PSA concentration in semen was approximately 2.5 mg/ml (between 0.5 and 5 mg/ml) then a 1 in 200 dilution was equivalent of 12.5 μg/ml (5 ml). These levels of PSA were considerably higher than the levels of PSA in serum, so a higher dilution of 1 in 100,000 was tested, in which the PSA concentration was approximately 25 ng/ml (5 ml). FIG. 5 shows the results of running the fractions on SDS-PAGE.

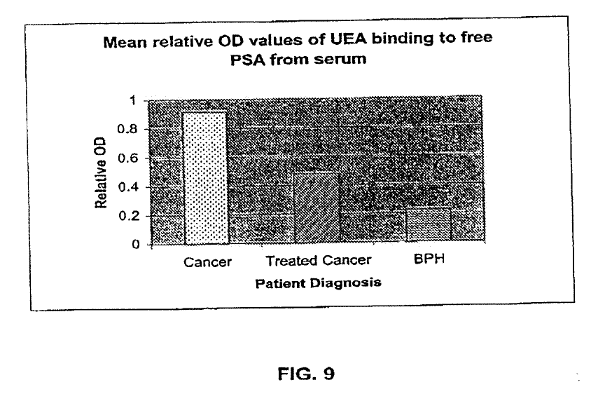

FIG. 5 shows the results of staining semen diluted 1 in 100,000. FIG. 5a shows the results of staining with silver stain, whilst FIG. 9b shows the results of staining with anti-PSA stain. Lanes 1 and 12 contain molecular weight markers; Lanes 2-11 contain the eluted PSA fractions 1 to 10.

FIG. 5a shows a band at 30 kDa (see black arrow), which was confirmed as being free PSA by the anti-PSA stain (see black arrow). The extent of non-specific binding was considerably lower, due to the higher dilution of the semen. FIG. 5b shows the PSA bands and elucidates clipped forms that are in such small quantities (i.e. <0.1 μg) that they were not identified with the silver stain. These results indicated that the columns were successful in isolating very small quantities of PSA from semen.

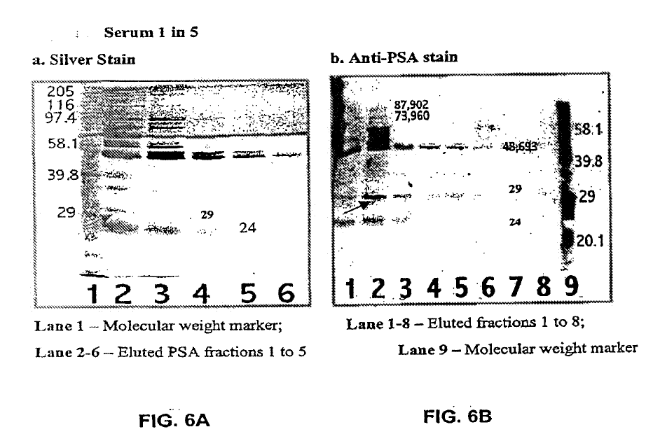

The columns were then tested with serum from a healthy male, where the PSA levels had been artificially raised to 1.5 ng/ml. FIG. 6 shows the results of running the fractions on SDS-PAGE.

FIG. 6 shows the results of staining serum diluted 1 in 5. FIG. 6a shows the results of staining with silver stain; Lane 1 contains molecular weight markers and Lanes 2-6 contain eluted PSA fractions 1 to 5. FIG. 6b shows the results of staining with anti-PSA stain. Lane 9 contains molecular weight markers and Lanes 1-8 contain the eluted PSA fractions 1 to 10.

FIG. 6a showed a faint band at ˜29 kDa (see black arrow), which was identified as PSA by the anti-PSA stain (FIG. 6b). A more intense band was seen just below the PSA band at ˜24 kDa in the silver stain and the ECL, this possibly represents a clipped isoform of PSA. Clusters of bands seen between 50 and 100 kDa on the anti-PSA stain are most probably PSA molecules in complex with other serum proteins. The first two fractions (lanes 2 and 3) of the silver stain (FIG. 6a) showed some non-specific binding of high molecular weight proteins. They do not cause a major problem as they do not appear to be masking or interfering with the free form PSA at 30 kDa, which is the protein of interest in this study.

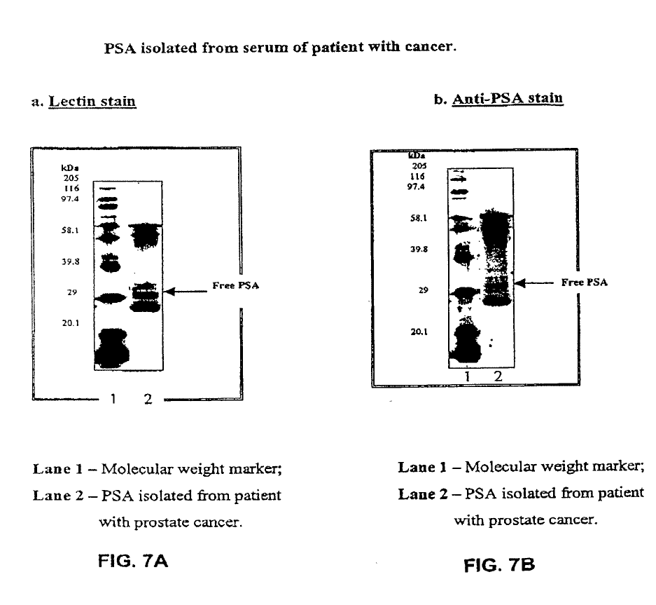

UEA binding to PSA; PSA was isolated from sera collected from patients with prostate cancer and BPH. The fractions were run on SDS-PAGE to check the PSA was isolated and non-specific binding was kept to a minimum. The fractions with the greatest quantity of PSA were re-run on SDS-PAGE and blotted to nitrocellulose then stained with the lectin UEA. FIGS. 7 and 8 demonstrate examples of Ulex binding strongly to free PSA isolated from a patient with cancer and also Ulex binding weakly to free PSA isolated from a patient with BPH.

FIG. 7 shows the results of staining PSA isolated from serum of patient with cancer. FIG. 7a shows the results of staining with lectin stain, whilst FIG. 7b shows the results of staining with anti-PSA stain. In both figures, Lane 1 contains molecular weight markers and Lane 2 contains PSA isolated from a patient with prostate cancer.

TABLE 1
Band intensity values for fPSA from cancer serum (FIG. 7).

| | Average intensity |
|---|---|
| | of free PSA |
| UEA | 0.901 |
| PSA | 0.881 |
| Relative Intensity | 1.02 |

FIG. 8 shows the results of staining PSA isolated from serum of patient with BPH. FIG. 8a shows the results of staining with lectin stain, whilst FIG. 7b shows the results of staining with anti-PSA stain. In both figures, Lane 1 contains molecular weight markers and Lane 2 contains PSA isolated from a patient with prostate cancer. Table 2 compares the band intensity values in FIG. 8.

TABLE 2
Band intensity values for fPSA from benign serum (FIG. 8).

| | Average intensity |
|---|---|
| | of free PSA |
| UEA | 0.0914 |
| PSA | 0.719 |
| Relative Intensity | 0.127 |

The X-rays were scanned using a GS-710 densitometer and the molecular weight and band intensity was calculated using Quantity One software (Biorad). The intensity of the free PSA band was measured from the lectin stain and was divided by the intensity of the free PSA band measured from the anti-PSA stain, thus giving a comparative value. The sera was grouped according to the diagnosis of the patient at the time the serum was taken, as either having prostate cancer or benign prostatic hyperplasia it was also noted whether the patient had received any treatment for his condition, had metastasis, or PIN. The actual measurements for intensity are shown for these examples (tables 1 and 2).

FIG. 9 shows the mean relative intensity of UEA/PSA for the prostate cancer group, BPH patients, and those who have received treatment for their prostate cancer.

The mean intensity of binding to free PSA from cancer patients was 0.9153 with a standard deviation of 0.205 (standard error of 7.25E-02) and the mean for the benign group was 0.2243 with a standard deviation of 0.1596 (standard error of 7.98E-02). A T-Test carried out showed this increase in binding to free PSA from cancer patients was highly significant (p=0.05).

What is claimed is:

1. A method for the detection of a cancerous condition in the prostate gland of a male human subject, said method comprising:
obtaining a sample from the subject;
substantially removing cells and cell debris from said sample,
assaying for the presence of a glycosylated protein uniquely associated with the prostate in said sample;
and, where said protein is present in the sample, comparing fucosylation thereof with a standard control value indicative of a male human subject having a normal, non-cancerous prostate gland;
a level of fucosylation in the sample which is statistically significantly greater than that of the control being taken as indicative of said cancerous condition.

2. The method of claim 1, wherein the condition is prostate carcinoma.

3. The method of claim 1, wherein the protein is a secreted protein.

4. The method of claim 1, wherein the protein is Prostate Specific Antigen.

5. The method of claim 1, wherein said sample is blood.

6. The method of claim 1, wherein said sample is semen.

7. The method of claim 1, wherein said sample is urine.

8. The method of claim 1, wherein said sample is centrifuged to remove any cellular material prior determination of fucosylation.

9. The method of claim 1, wherein fucosylation is determined using a lectin.

10. The method of claim 9, wherein the lectin is Ulex lectin.

11. The method of claim 9, wherein the lectin is labeled.

12. The method of claim 1, wherein fucosylation is determined using a monoclonal antibody specific for said protein when exhibiting elevated levels of fucose.

13. The method of claim 12, wherein said antibody is labeled.

14. The method of claim 11, wherein the label is selected from the group consisting of fluorophores and radio-isotopes.

15. The method of claim 13, wherein the label is selected from the group consisting of fluorophores and radio-isotopes.

16. A kit for testing a sample derived from a mammal comprising means for detecting PSA having an abnormal fucose content.

17. A kit for a assaying a sample obtained from a male human subject, comprising:
means for determining the presence of a glycosylated protein uniquely associated with the prostate in said sample;
means for determining fucosylation of said glycosylated protein;
and means for comparing fucosylation determined with a standard control value representative of a male human subject having a normal, non-cancerous prostate gland.

18. The kit of claim 17, wherein the glycosylated protein is Prostate Specific Antigen.

19. The kit of claim 17, wherein said sample is blood serum.

20. The kit of claim 17, wherein said sample is semen.

21. The kit of claim 17, wherein said sample is urine.

22. The kit of claim 17, wherein the means for determining fucosylation is a lectin.

23. The kit of claim 22, wherein the lectin is Ulex lectin.

24. The kit of claim 22, wherein the lectin is labeled.

25. The kit of claim 17, wherein the means for determining fucosylation is a monoclonal antibody specific for said protein when exhibiting elevated levels of fucose.

26. The kit of claim 25, wherein said antibody is labeled.

27. The kit of claim 24, wherein the label is selected from the group consisting of fluorophores and radio-isotopes.

28. The kit of claim 26, wherein the label is selected from the group consisting of fluorophores and radio-isotopes.

29. The kit of claim 17, wherein the means of fucosylation is evidenced by a color change.

30. The kit of claim 29, wherein the color change is due to an enzyme reaction.

31. A kit of claim 29, wherein the color change is evaluated by the eye.

32. The kit of claim 29, wherein the color change is measured mechanically.

33. A method for the detection of prostate carcinoma in a male human subject, said method comprising:
obtaining a sample of bodily fluid from the subject;
substantially removing cells and cell debris from the sample by centrifugation;
assaying for the presence of Prostate Specific Antigen (PSA) in the sample;
and, where PSA is present in the sample, comparing fucosylation thereof with a standard control value indicative of PSA from a male human subject having a normal, non-cancerous prostate gland;
a level of fucosylation of PSA in the sample which is statistically significantly greater than that of the control being taken as indicative of the cancerous condition.

34. The method of claim 33, wherein the sample is blood.
